(19) United States
(12) Patent Application Publication
(10) Pub. No.: US 2007/0202565 A1
(43) Pub. Date: Aug. 30, 2007

(54) Growth plotter

(76) Inventors: Debra J. Fleury, Lee's Summit, MO (US); Todd A. Fleury, Lee's Summit, MO (US)

Correspondence Address: HARSHAW RESEARCH, INC., 210 W. TECUMSEH STREET, OTTAWA, KS, 66067, US

(21) Appl. No.: 11/363,327

(22) Filed: Feb. 27, 2006

(51) Int. Cl. C12Q 1/20

(52) U.S. Cl. 435/33

(57) ABSTRACT

A digital growth plotter according to the present invention includes a housing for being conveniently and ergonomically held by a caregiver. The growth plotter includes a processor, input devices, a data storage device, and a display. The housing may include a battery for energizing the electronic components. The input device may include a keypad and touch screen for efficient user entry of growth information, said growth information being stored in the digital storage device. The processor includes programming for downloading an up-to-date growth chart for comparison with recorded growth information. The processor also includes programming for plotting a growth chart according to the recorded and standard growth information. Then, the processor may upload the recorded information to a compiling entity so that the standard chart may be updated/revised for more accurate comparisons and analysis in the future.

### BACKGROUND OF THE INVENTION

This invention relates generally to growth charts and, more particularly, to an electronic growth plotter for pediatricians to gather, record, and plot growth data for their patients. The growth plotter also provides updated data directly to a compiling organization, such as the Center for Disease Control (“CDC”), for contribution to the compilation of up-to-date growth charts.

In 1977, the National Center for Health Statistics developed growth charts for assisting pediatricians in determining if a patient's growth is adequate. The Center for Disease Control revises these charts based on submissions of new data. These charts can only be revised, of course, as new data is submitted. While various devices have been proposed for measuring and recording patient growth information, these devices and systems do not provide adequate or efficient means for collecting, recording, and immediately plotting growth information or for immediately being electronically submitted to the compiling organization for updating the standard growth charts.

Therefore, it would be desirable to have an electronic growth plotter which enables a health professional to conveniently measure and record many types of health and growth data. Further, it would be desirable to have an electronic growth plotter which can both download current/standard CDC growth charts and can upload patient data to the CDC for the generation of updated standard growth charts. In addition, it would be desirable to have an electronic growth plotter which can update and print growth chart data specific to a patient.

### SUMMARY OF THE INVENTION

Accordingly, a digital growth plotter according to this invention includes a housing, an input device, and a processor positioned in the housing. The input device may include a keypad and selector buttons on the housing that are in electric communication with the processor and a data storage device. The input device may also include a touch screen for entering predetermined types of health and growth data. The processor includes programming for comparing recorded growth information to a growth chart previously stored in the storage device. The processor further includes programming and circuitry for retrieving a growth chart from a remote computer and for uploading collected growth information to the remote computer so that it can be used in revising the growth chart. Examples of growth data to be recorded, tracked, and shared, may include the sex of the patient, height, weight, and other similar statistics.

Therefore, a general object of this invention is to provide a digital growth plotter for recording and plotting patient growth data.

Another object of this invention is to provide a digital growth plotter, as aforesaid, which can download up-to-date growth charts from a remote computer using a computer network.

Still another object of this invention is to provide a digital growth plotter, as aforesaid, which can upload presently recorded data to a remote computer such as the CDC for immediate use in revising standard growth charts.

Yet another object of this invention is to provide a digital growth plotter, as aforesaid, which can plot the recorded growth information.

A further object of this invention is to provide a digital growth plotter, as aforesaid, which can compare the plotted recorded growth information with the plotted growth chart received from the remote computer.

A still further object of this invention is to provide a digital growth plotter, as aforesaid, which an input device for obtaining growth data includes a touch screen.

Other objects and advantages of this invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a digital growth plotter according to a preferred embodiment of the present invention;

FIG. 2a is a front view of a housing of the growth plotter as in FIG. 1a;

FIG. 2b is a sectional view taken along line 1c-1c of FIG. 1b;

FIG. 3 is a flowchart illustrating the logic performed by the processor of the growth plotter according to the present invention;

FIG. 4 is a block diagram illustrating the electronic components of the present invention; and

FIG. 5 is a plotted growth chart of recorded growth data in comparison to a plotted standard growth chart.

### DESCRIPTION OF THE PREFERRED EMBODIMENT

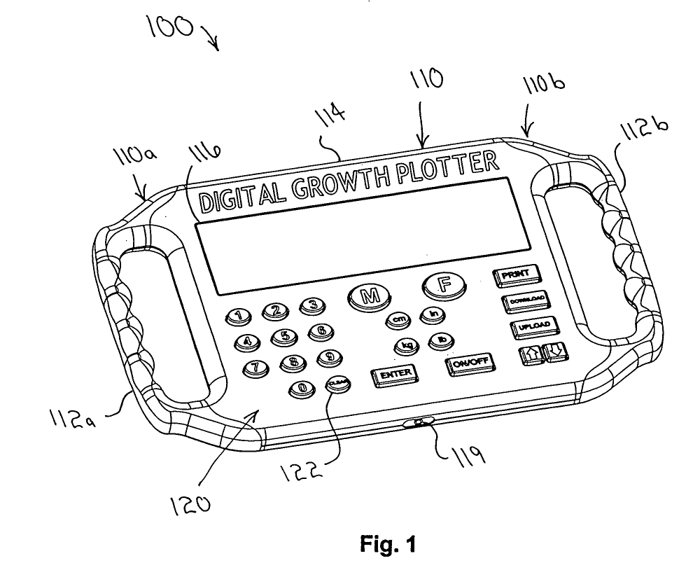

A digital growth plotter 100 according to the present invention will now be described in detail with reference to FIGS. 1 through 5 of the accompanying drawings. More particularly, a growth plotter 100 according to the current invention includes a housing 110, an input device 120, and a processor 130 secured inside the housing 110.

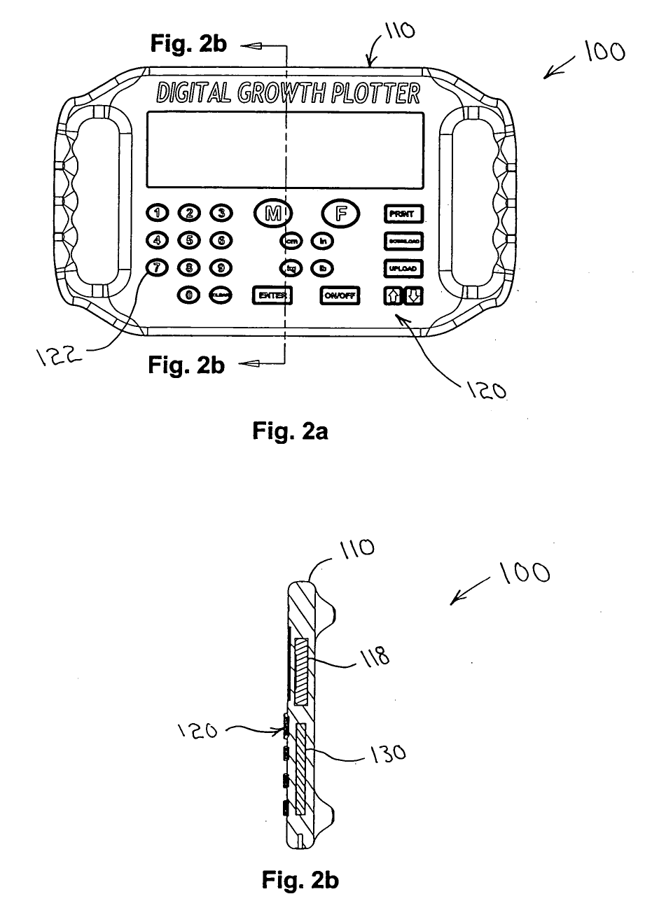

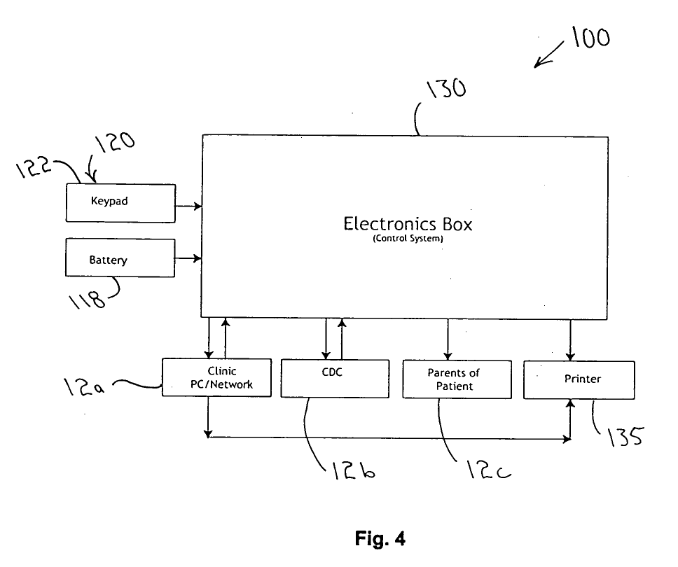

As shown in FIG. 1, the housing 110 may present opposed first and second ends 110a, 110b. A first handle 112a may be formed at the first end 110a, and a second handle 112b may be at the second end 110b. The handles 112a, 112b may be ergonomic handles configured to receive individual fingers and constructed of a soft elastomeric material or another ergonomic material. A central portion 114 of the housing 110 may include a durable plastic material or another appropriate material for protecting the processor 130. The input device 120 may include a keypad 122 (FIGS. 1 and 4), a touchscreen, a pointer (e.g., a touchpad, mouse, trackball, etc.), and/or another data-inputting device, and the input device 120 is preferably in communication with the processor 130 (FIG. 4). The housing 110 may include a display 116 that is also in communication with the processor 130. A power supply 118 (e.g., a rechargeable battery) may be electrically connected to the display 116 and the processor 130 for energizing the display 116 and the processor 130, respectively (FIG. 4). FIG. 2b shows the processor 130 and the battery 118 inside the housing 110. The battery 118 may be recharged by connecting an in/out port 119 to a computer or a power source (e.g., a 110-volt AC power outlet), or through other charging methods.

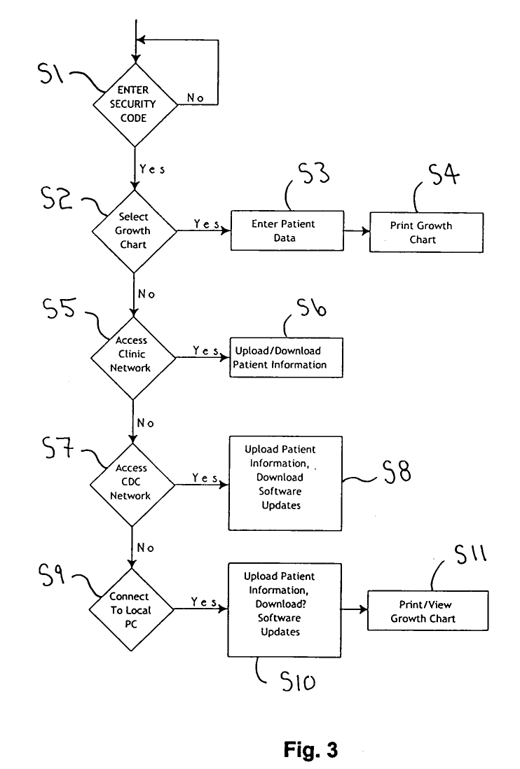

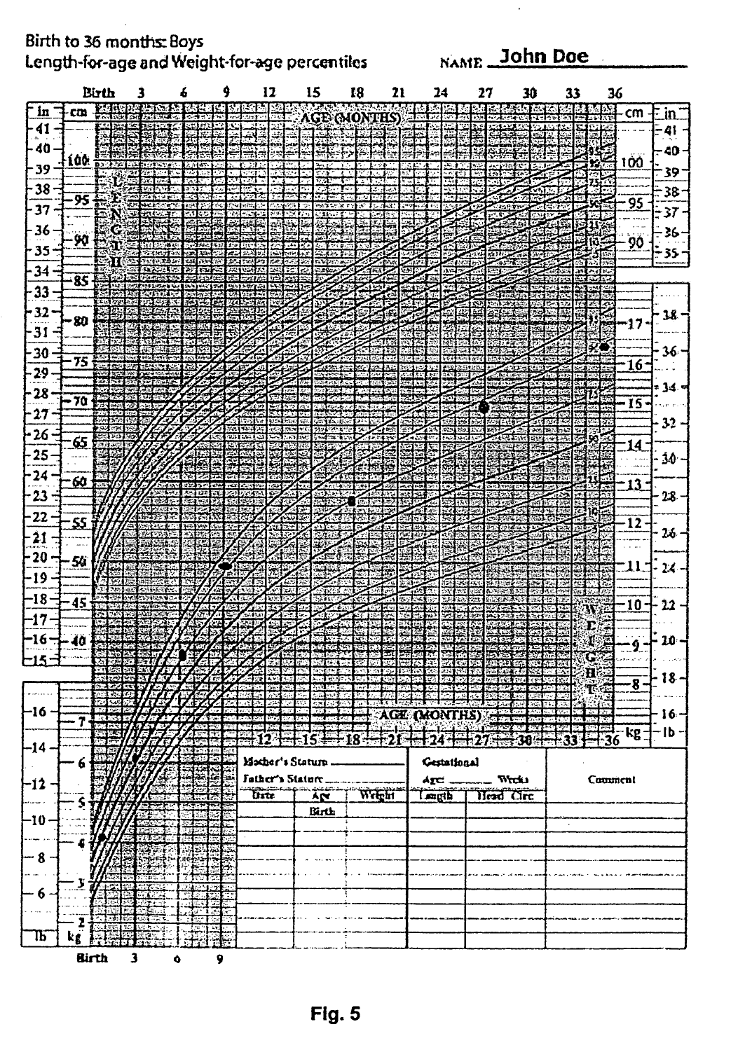

The processor 130 may have programming (that includes commands) for performing, and may actually perform, various functions, some of which are illustrated by FIG. 3. For example, the processor 130 may require a code (e.g., a security code or a patient identification code) to be input at step S1. The processor 130 may then allow a user to select a data entry mode at step S2. If the user does select the data entry mode at step S2, the processor 130 may request growth information to be input through the input device 120, and the user may actually provide the growth information through the input device 120 at step S3. As the user provides the growth information at step S3, the processor 130 may record the growth information (e.g., to a data storage device in communication with the processor 130). The processor 130 may then compare the recorded growth information to a standard growth chart 10 stored in the data storage device and print a plot of the recorded growth information and the growth chart at step S4, so that plotted recorded growth information may be visually compared to the plotted growth chart. An example printout of plotted recorded growth information and a standard growth chart is shown in FIG. 5. The processor 130 may alternately or additionally include programming for actuating the printer 135 (FIG. 4) to print the recorded growth information in an unplotted (i.e., non-graphical) format.

As shown at steps S5, S7, and S9 of FIG. 3, the processor 130 may selectively access various remote computers through a network (e.g., a local network or a wide area network such as the internet). At step S5, the user may choose to access a computer 12a (FIG. 4) that is accessible by a physician for monitoring a patient's growth. As shown at step S6, the processor 130 may include programming for selectively providing the growth information recorded at step S3 (discussed above) to the computer 12a, and also programming for selectively receiving information from the computer 12a.

At step S7, the user may choose to access a computer 12b (FIG. 4) that is accessible by a compiling entity for updating the standard growth chart used by physicians. The compiling entity may be the Center for Disease Control (the CDC), another government entity, or a private entity, for example. As shown at step S8, the processor 130 may include programming for selectively providing the growth information recorded at step S3 (discussed above) to the computer 12b, and also programming for selectively receiving information from the computer 12b. This step enables recorded growth information of a patient to be given to the compiling, e.g. the CDC, for use in compiling accurate revised standard growth charts. The revised growth chart, then, may be received from the computer 12b for use in analyzing the next patient's growth information. The processor 130 may further include programming for updating the standard growth chart upon receiving the updated standard growth chart information from the computer 12b.

At step S9, the user may choose to access a computer 12c (FIG. 4) that is accessible by a patient. “Patient” as used herein also includes a patient's caregiver (e.g., parents, etc.), as the patient will often be an infant or adolescent. As shown at step S10, the processor 130 may include programming for selectively providing the growth information recorded at step S3 (discussed above) to the computer 12c, providing the standard growth chart (and especially the growth chart updated at step S8 above) to the computer 12c, and also programming for selectively receiving information from the computer 12c. The patient may then print or view the growth chart and the recorded growth information as shown at step S11.

It is understood that while certain forms of this invention have been illustrated and described, it is not limited thereto except insofar as such limitations are included in the following claims and allowable functional equivalents thereof.

What is claimed is as follows:

1. A growth plotter, comprising:
a housing;
an input device; and
a processor secured inside said housing and having programming for requesting and recording growth information and selectively providing said recorded growth information to a remote computer through a network.

2. The growth plotter of claim 1, wherein:
a data storage device is in communication with said processor; and
said programming includes commands for comparing said recorded growth information to a growth chart stored in said data storage device.

3. The growth plotter of claim 2, wherein said processor includes programming for updating said growth chart from information received from said remote computer.

4. The growth plotter of claim 3, wherein said processor includes programming for plotting said recorded growth information and plotting said growth chart so that said plotted recorded growth information may be visually compared to said plotted growth chart.

5. The growth plotter of claim 1, wherein said input device includes a keypad.

6. The growth plotter of claim 1, wherein said input device includes a touchscreen.

7. The growth plotter of claim 1, wherein:
said housing includes first and second handles;
said first handle is at a first end of said housing;
said second handle is at a second end of said housing; and
said first and second ends of said housing are opposed.

8. The growth plotter of claim 7, wherein said first and second handles are ergonomic handles.

9. A growth plotter, comprising:
a housing;
an input device; and
a processor secured inside said housing and performing functions including:
requesting growth information;
recording growth information; and
selectively providing recorded growth information to at least one remote computer.

10. The growth plotter of claim 9, wherein one said remote computer is a computer that is accessible by a compiling entity for updating a standard growth chart.

11. The growth plotter of claim 10, wherein another said remote computer is a computer that is accessible by a physician for monitoring a patient's growth.

12. The growth plotter of claim 11, wherein yet another said remote computer is a computer that is accessible by a patient.

13. The growth plotter of claim 12, wherein said processor performs additional functions, including providing said standard growth chart to said remote computer that is accessible by a patient.

14. The growth plotter of claim 9, wherein said processor performs additional functions, including actuating a printer to print said recorded growth information.

15. The growth plotter of claim 9, wherein said processor performs additional functions, including comparing said recorded growth information to a standard growth chart.

16. The growth plotter of claim 15, wherein said processor performs additional functions, including updating said standard growth chart from information received from one said remote computer.

17. The growth plotter of claim 15, wherein said processor performs additional functions, including actuating a printer to print said comparison of said recorded growth information and said standard growth chart.

18. The growth plotter of claim 9, further comprising:
a display; and
a battery electrically connected to said processor and said display for energizing said processor and said display, respectively.

19. The growth plotter of claim 9, wherein said input device is selected from the group consisting of a keypad, a touchscreen, and a pointer.

20. The growth plotter of claim 19, wherein:
said housing includes first and second handles;
said first handle is at a first end of said housing;
said second handle is at a second end of said housing; and
said first and second ends of said housing are opposed.
